(19) United States
(12) Patent Application Publication
(10) Pub. No.: US 2025/0018122 A1
(43) Pub. Date: Jan. 16, 2025

(54) PLUNGER ROD REMOVAL FORCE METHOD AND FIXTURE

(71) Applicant: AMGEN INC., Thousand Oaks, CA (US)

(72) Inventors: Ryan Oba, Santa Paula, CA (US); Mher Asadourian, Glendale, CA (US); Sandra Sanchez, Thousand Oaks, CA (US); Chelsea Balli-Cruz, Winnetka, CA (US); Joshua Tamsky, Los Angeles, CA (US)

(21) Appl. No.: 18/710,112

(22) PCT Filed: Nov. 28, 2022
(86) PCT No.: PCT/US22/51036

§ 371 Date: May 14, 2024

Related U.S. Application Data
(60) Provisional application No. 63/283,798, filed on Nov. 29, 2021.

(51) Int. Cl. A61M 5/31; G01M 99/00

(52) U.S. Cl. CPC A61M 5/3129; G01M 99/005

(57) ABSTRACT

A testing system for a drug delivery device includes a frame member having first and second ends, a driving member positioned near the frame member, a surrogate syringe barrel operably coupled with the frame member, a flange extender adapted to couple with a portion of the surrogate syringe barrel, and a plunger rod adapted to couple with the flange extender and be at least partially disposed within the surrogate syringe barrel while being movable therein. The driving member is movable in an axial direction between the first end and the second end of the frame member. The plunger rod includes first and second ends and a longitudinal length therebetween, the first end being positioned near a portion of the driving member. The driving member measures a maximum urging force exerted on the plunger rod when moving in the axial direction towards the second end of the frame member.

### CROSS-REFERENCE TO RELATED APPLICATION

Priority is claimed to U.S. Provisional Patent Application No. 63/283,798, filed Nov. 29, 2021, the entire contents of which are hereby incorporated by reference herein.

### FIELD OF DISCLOSURE

The present disclosure generally relates to drug delivery devices and, more particularly, to retention features for drug delivery devices.

### BACKGROUND

Drug delivery devices, such as injectors, are used to deliver liquid drugs to a patient. Upon activation, a drug delivery device will expel a drug stored within an internal reservoir such as, for example, a pre-filled syringe (“PFS”) through a needle, cannula, or other delivery member into the patient. Some drug delivery devices, such as pen-type autoinjectors or on-body injectors, may be positioned adjacent to a patient's skin to deliver a drug via an injection needle or some other means over a period of time. The drug delivery device may be positioned near the tissue of the patient's abdomen, thigh, arm, or some other portion of the patient's body.

To ensure patient and/or user safety, these devices undergo any number of sterilization steps to ensure unwanted contaminants do not contact specific components of the device and/or the drug itself. One such example of a sterilization approach is commonly known as “external sterilization” which includes placing a partially or fully assembled drug delivery device into a sterilization chamber and subjecting the device to a sterilizing gas or other sterilizing agent such as, for example nitrogen dioxide sterilization, ethylene oxide sterilization, light-based sterilization, and/or any other suitable approaches. In many of these processes, during the external sterilization process, a vacuum is applied to the sterilization prior to the introduction of the sterilant gas in order to maximize contact between the sterilant gas and the external surface of the fully and/or partially assembled and filled device. When the vacuum is applied, the resulting pressure differential between the interior and the exterior of the PFS causes an air bubble within the PFS to expand, which may in turn urge or move additional components of the device (e.g., a plunger stopper) in an undesired manner.

Some drug delivery devices may include features that resist and/or limit the motion of the plunger stopper, as such movement may result in previously sterilized components entering non-sterile regions. Further, such movement may impact performance of the device upon administration of the drug. Existing testing approaches attempted to measure the force required to overcome such retention features, but oftentimes yield inaccurate results due to not properly replicating the forces encountered during the sterilization process.

The present disclosure sets forth plunger rod removal force fixtures and corresponding approaches embodying advantageous alternatives to existing fixtures and approaches, and that may address one or more of the challenges or needs mentioned herein.

### SUMMARY

In accordance with a first aspect, a testing system for a drug delivery device includes a frame member having first and second ends, a driving member positioned near the frame member, a surrogate syringe barrel operably coupled with the frame member, a flange extender adapted to couple with a portion of the surrogate syringe barrel, and a plunger rod adapted to couple with the flange extender and be at least partially disposed within the surrogate syringe barrel while being movable therein. The driving member is movable in an axial direction between the first end and the second end of the frame member. The plunger rod includes first and second ends and a longitudinal length therebetween, the first end being positioned near a portion of the driving member. The driving member measures a maximum urging force exerted on the plunger rod when moving in the axial direction towards the second end of the frame member.

In some examples, the plunger rod may include a projecting/protruding plunger rod interface feature positioned along the longitudinal length thereof. The plunger rod interface may engage a floor region of the flange extender to retain the plunger rod in an initial position. In some of these approaches, the plunger rod interface may include a ring having an outer dimension. Further, in these and other examples, the floor region of the flange extender may include an opening having an inner dimension. The outer dimension of the ring of the plunger rod may be greater than the inner dimension of the opening of the flange extender. In these arrangements, axial movement of the plunger rod towards the second end of the frame member is prevented. In some examples, at least one of the plunger rod interface or the floor region of the flange is at least partially constructed from a deformable material.

In some approaches, the surrogate syringe barrel may include a syringe flange. The flange extender may include a flange adapted to rest upon the syringe flange of the surrogate syringe barrel.

In these and other examples, the frame member may include an elevated platform positioned at the first end thereof. The elevated platform may retain and suspend the surrogate syringe barrel from the first end thereof. In these examples, when moving the driving member towards the second end of the frame member, the plunger rod and the flange extender are subject to a gravitational force in the axial direction

In some approaches, the driving member includes a push pin that is at least partially insertable and movable within the surrogate syringe barrel. The push pin may include a recessed region that aligns with the first end of the plunger rod.

In some approaches, the driving member may be positioned above the first end of the frame member.

In accordance with a second aspect, an approach for measuring a maximum urging force experienced by a portion of a drug delivery device is provided that includes positioning a driving member above a first end of a frame member. The driving member is movable in an axial direction between the first end of the frame member to a second end of the frame member. The approach further includes operably coupling a surrogate syringe barrel with the first end of the frame member, coupling a first flange extender with a portion of the surrogate syringe barrel and coupling a first plunger rod with the flange extender such that the first plunger rod is at least partially movably disposed within the surrogate syringe barrel. The first plunger rod includes a first end, a second end, and a longitudinal length therebetween. The approach further includes moving the driving member in the axial direction towards the second end of the frame member, thereby urging the first plunger rod towards the second end of the frame member. A maximum urging force exerted on the first plunger rod while moving towards the second end of the frame member is measured.

### BRIEF DESCRIPTION OF THE DRAWINGS

The above needs are at least partially met through provision of the plunger rod removal force method and fixture described in the following detailed description, particularly when studied in conjunction with the drawings, wherein:

FIG. 1 illustrates an example testing system in accordance with various embodiments;

FIG. 2 illustrates the example testing system of FIG. 1 prior to initiating testing in accordance with various embodiments;

FIG. 3 illustrates a bottom view of an example elevated platform for use with the example testing system of FIGS. 1 and 2 in accordance with various embodiments;

FIG. 4 illustrates the testing system of FIGS. 1-3 in a pre-test configuration in accordance with various embodiments;

FIG. 5 illustrates a close-up view of a portion of the drug delivery device in the example testing system of FIGS. 1-4 in accordance with various embodiments; and

FIG. 6 illustrates the example portion of the drug delivery device in the example testing system of FIGS. 1-5 upon completion of testing in accordance with various embodiments.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments. It will further be appreciated that certain actions and/or steps may be described or depicted in a particular order of occurrence while those skilled in the art will understand that such specificity with respect to sequence is not actually required. It will also be understood that the terms and expressions used herein have the ordinary technical meaning as is accorded to such terms and expressions by persons skilled in the technical field as set forth above except where different specific meanings have otherwise been set forth herein.

### DETAILED DESCRIPTION

Generally speaking, pursuant to these various embodiments, an approach is provided that accurately simulates a load experienced by the drug delivery device during interactions within a sterilization chamber. The approach includes a testing system that urges delivery device components in a direction that mirrors the forces experienced by the components during sterilization processes. More specifically, the approach simulates forces experienced by the drug delivery device during an external sterilization process, which, as previously noted, includes placing a partially or fully assembled drug delivery device (that, in some examples, may be filled with a drug product) into a sterilization chamber and subjecting the device to a sterilizing gas such as, for example nitrogen dioxide sterilization, ethylene oxide sterilization, light-based sterilization, and/or any other suitable approaches. The testing system measures a maximum force experienced by the device components to assist with assessing their potential use in varying drug delivery device designs.

The drug delivery device delivers a drug, which may also be referred to herein as a medicament or drug product. The drug may be, but is not limited to, various biologicals such as peptides, peptibodies, or antibodies. The drug may be in a fluid or liquid form, although the disclosure is not limited to a particular state. Various implementations and configurations of the drug delivery device are possible. For example, the present disclosure describes a drug delivery device in the form of a single-use, disposable injector. In other embodiments, the drug delivery device may be configured as multiple-use reusable injector. The drug delivery device may be operable for self-administration by a patient or for administration by a caregiver or a formally trained healthcare provider (e.g., a doctor or nurse). Further, the drug delivery device may take the form of an autoinjector or pen-type injector, and, as such, may be held in the hand of the user over the duration of drug delivery or dosing.

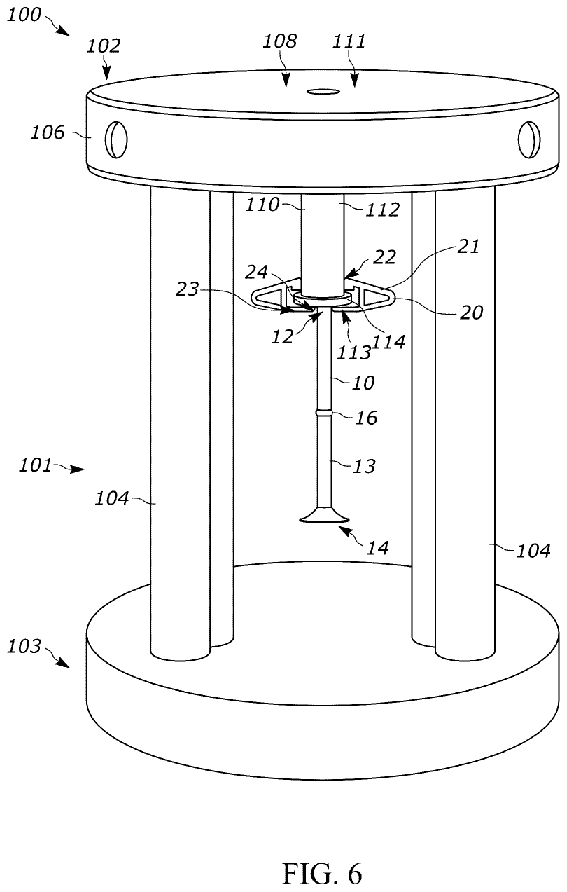

Turning now to the Figures, a testing system 100 is provided for use with components of a drug delivery device. More specifically, a plunger rod 10 is provided that couples with a flange extender 20. Generally speaking, when used for drug delivery, the plunger rod 10 is disposed within or near a syringe barrel (not illustrated) containing a drug (not illustrated) to be administered to a user. The plunger rod 10 is movable within the syringe barrel in an axial direction to urge a plunger towards a delivery member (e.g., a needle and/or a cannula) that pierces a user's skin to deliver the drug or medicament into the patient's tissue. The plunger rod 10 includes a first end 12 (FIG. 6) which may be coupled with a plunger via any number of suitable approaches, a second end 14, and a longitudinal length 13 therebetween. As illustrated in FIG. 6, the plunger rod 10 further includes a projecting and/or protruding plunger rod interface 16 (FIG. 6) positioned along the longitudinal length 13. In the illustrated example, the plunger rod interface 16 is in the form of a ring that extends around an outer periphery of the longitudinal length 13. The ring 16 may have an outer dimension (e.g., diameter). It is appreciated that the plunger rod 10 may include any number of additional features (e.g., additional interfaces, guiding mechanisms, coupling mechanisms, and the like) to assist with operation of the drug delivery device. In some examples, all or a portion of the plunger rod 10 is constructed from a resilient material such as, for example, a polymeric material. Other examples are possible.

The flange extender 20 is adapted to couple with the plunger rod 10 during use of the drug delivery device. Generally speaking, the flange extender 20 may provide a user with an increased surface area to grasp before, during, and/or after drug administration. The flange extender 20 includes a body 21 defining an upper opening 22, a floor region 23, and a lower opening 24 extending through the floor region 23, and further includes a flange 26 extending downwardly into the body 21 that at least partially defines the upper opening 22. The lower opening 24 defines an inner dimension. In some examples, all or a portion of the flange extender 20 is constructed from a resilient material such as, for example, a polymeric material. Other examples are possible.

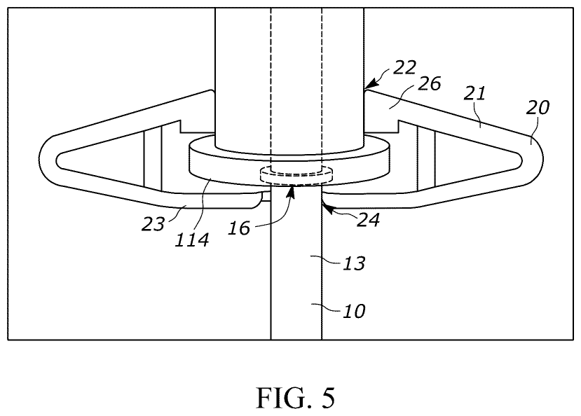

The plunger rod 10 is movable in the axial direction (i.e., along the longitudinal length 13 of the plunger rod 10) relative to the flange extender 20. Further, a portion of the longitudinal length 13 of the plunger rod is insertable through the upper opening 22 and/or the lower opening 24 of the flange extender 20. Notably, and as illustrated in FIG. 5, before the drug delivery device is used, and prior to sterilization, the ring 16 of the plunger rod 10 is positioned above the lower opening 24 of the flange extender 20. In this example, the outer dimension of the ring 16 is greater than the inner dimension of the lower opening 24 of the flange extender 20 (though the remainder of the longitudinal length 13 of the plunger rod 10 has a smaller dimension than the inner diameter of the lower opening 24 of the flange extender 20). Accordingly, in this initial position, the plunger rod 10 is retained by the flange extender. This engagement restricts the plunger rod 10 from moving in the downward direction.

The interference between the ring 16 and the lower opening 24 may be overcome by sufficient urging in the axial direction. For example, depending on the characteristics of the plunger rod 10 and the flange extender 20, the ring 16 of the plunger rod may be constructed from a deformable material such that it compresses and/or otherwise deforms. Additionally and/or alternatively, the lower opening 24 of the flange extender 20 may be constructed from a deformable material such that it increases in size or otherwise deforms due to deformation of the body 21. In such examples, the engagement between the ring 16 of the plunger rod 10 and lower the opening 24 of the flange extender 20 may be defined by a retention force, that is, a maximum force exerted on these components before the ring 16 of the plunger rod 10 passes through the lower opening 24 of the flange extender 20. During the (external) sterilization phase where a partially and/or fully assembled device which may contain a drug product therein are subjected to a sterilant (as well as during other phases such as transport and other handling), the pressure differential between the interior and exterior of the syringe barrel may cause an air bubble within the syringe barrel to expand, thereby urging the plunger (and thus the plunger rod) downward. It is thus advantageous to identify the maximum retention force exhibited between the plunger rod 10 and the flange extender 20 in order to ensure that this maximum retention force is greater than the peak/maximum urging force generated by the gas bubble to confirm the plunger rod will not move beyond a desired location.

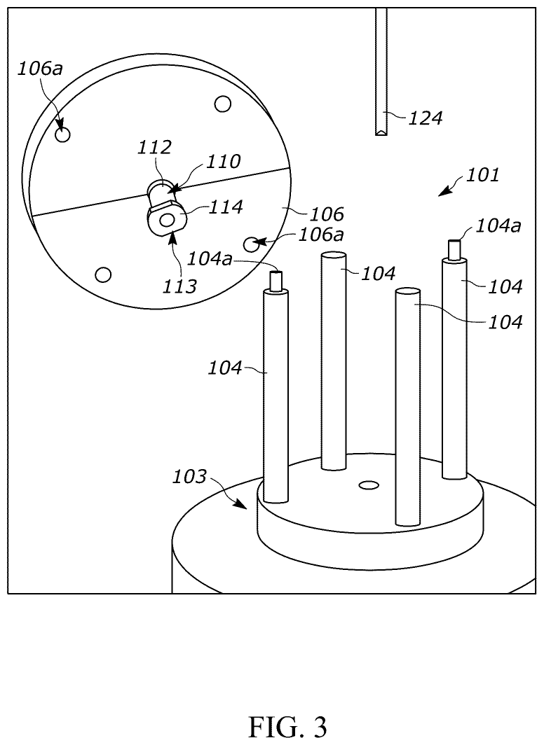

The test system 100 is arranged to test the engagement between the plunger rod 10 and the flange extender 20 and to measure a force that corresponds to the maximum retention force experienced prior to the ring 16 of the plunger rod 10 passing through the lower opening 24 of the flange extender 20. Importantly, the test system 100 is oriented in a way to simulate relative positioning of the components of the drug delivery device during various processes such as sterilization, and further applies a maximum urging force in the same direction as forces experienced by the device during these processes (i.e., “pushing” on the first end 12 of the plunger rod 10, rather than “pulling” on the second end 14 of the plunger rod 10). The test system 100 includes a frame member 101 having a first end 102 and a second end 103, a surrogate syringe barrel 110, and a driving member 120 positioned generally near (e.g., above) the first end 102 of the frame member 101 and the surrogate syringe barrel 110. In the illustrated examples, the first end 102 corresponds to an upper end of the frame member 101, and the second end 103 corresponds to a lower end thereof. Accordingly, these terms may be used interchangeably throughout the description. Further, it will be appreciated that other arrangements and/or orientations are possible. The lower end 103 of the frame member 101 may rest on a horizontal surface, or alternatively, may be suspended by a shaft or similar component. The frame member 101 includes at least one support column 104 coupled with an elevated platform 106 positioned at the upper end 102 thereof. As illustrated in FIG. 3, the support column or columns 104 may include one or more alignment pins 104a that is insertable within corresponding support opening(s) 106a formed on or in the platform 106. The platform 106 may further include an opening 108 configured to receive the surrogate syringe barrel 110.

The surrogate syringe barrel 110 is in the form of a generally hollow cylindrical member that replicates a syringe barrel of the drug delivery device. In the illustrated example, the surrogate syringe barrel 110 is a headless member that lacks a coupling point for the needle or other components of the drug delivery device. The surrogate syringe barrel 110 includes a first end 111, a second end 113, a body 112 disposed between the first and second ends 111, 113, and a syringe flange 114 positioned at the second end 113. In some examples, the needle of the drug delivery device would be disposed at the first end 111. The first end 111 of the surrogate syringe barrel 110 is coupled with the platform 106. More specifically, the first end 111 of the surrogate syringe barrel 110 may be disposed within the opening 108 of the platform 106. In some examples, the first end 111 of the surrogate syringe barrel 110 may frictionally engage the opening 108 of the platform 106, and in other examples, the platform 106 may be a two or multi-piece member that may be adjusted or tightened to clamp and secure the first end 111 of the surrogate syringe barrel 110 therein.

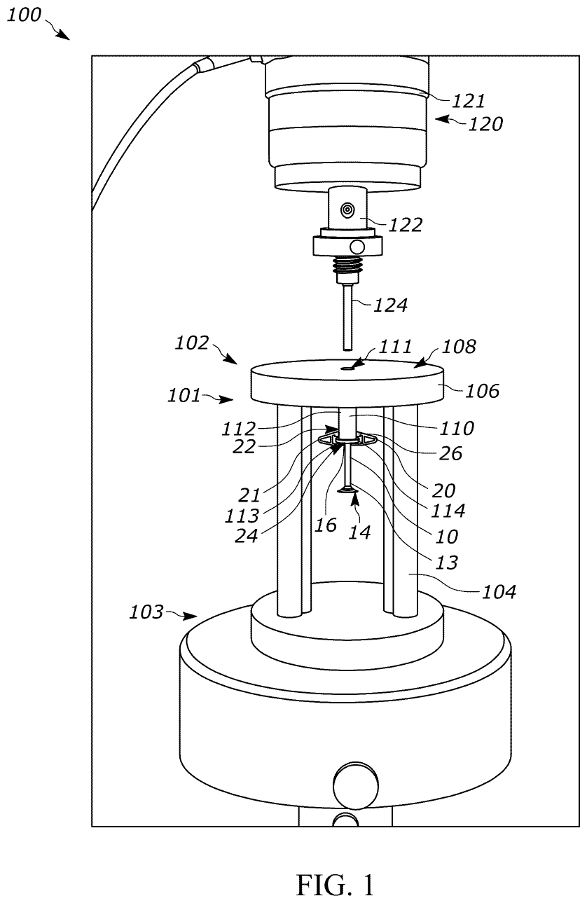

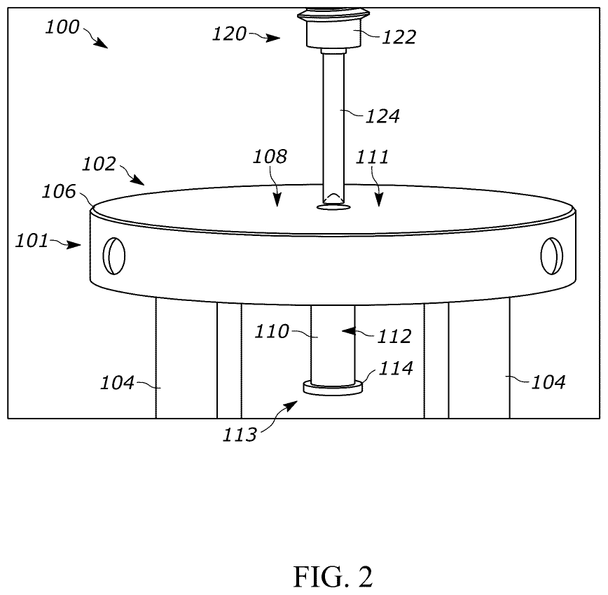

The driving member 120 is arranged to exert a force on the plunger rod 10. More specifically, the driving member 120 includes a load cell 121, a moveable coupling 122, and a push pin 124 operably coupled with the movable coupling 122. As illustrated in FIG. 1, the moveable coupling 122 includes a threaded receptacle that receives the push pin 124, exerts force on the plunger rod 10 during operation. The load cell 121 may be any type of suitable load cell capable of measuring a force exerted on the push pin 124. The movable coupling 122 moves in an axial direction from an initial, upper position (i.e., at or near the upper end 102 of the frame member 101) to a lowered position (i.e., in a direction towards the lower end 103 of the frame member 101). Because the push pin 124 is coupled therewith, it also moves in an axial direction. As illustrated in FIGS. 1 and 2, the push pin 124 is axially aligned with the surrogate syringe barrel 110 such that the push pin 124 may move axially therethrough.

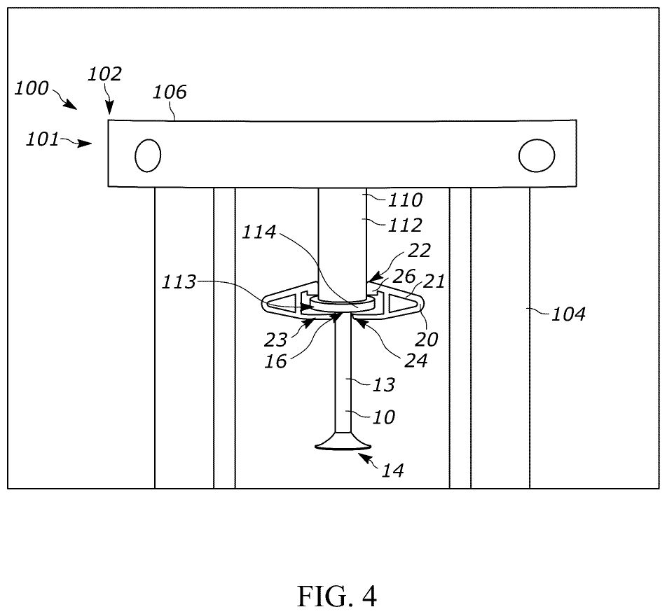

In use, upon coupling the surrogate syringe barrel 110 with the platform 106, the first end 111 of the surrogate syringe barrel 110 is axially aligned with the push pin 124. The first end 12 of the plunger rod 10 may be inserted into the second end 113 of the surrogate syringe barrel 110, and the flange extender 20 may be coupled with the second end 113 of the surrogate syringe barrel 110. More specifically, as illustrated in FIGS. 4-6, a portion of the body 112 of the surrogate syringe barrel 110 may be positioned through the upper opening 22 of the flange extender 20, while the flange 26 of the flange extender 20 rests on the syringe flange 114 of the surrogate syringe barrel 110. In this configuration, the syringe flange 114 of the surrogate syringe barrel 110 is positioned above the lower opening 24 of the flange extender 20, and as such, mimics the configuration of the drug delivery device in an assembled state. As previously noted, in this configuration, the ring 16 of the plunger rod 10 is also positioned above the lower opening 24 of the flange extender 20. In some examples, the ring 16 of the plunger rod 10 may be disposed completely and/or partially within the throughbore of the surrogate syringe barrel 110, and in other implementations, the ring 16 of the plunger rod 10 may be disposed at or near the syringe flange 114 of the surrogate syringe barrel 110. Further, in some examples, the first end 12 of the plunger rod 10 may extend upwards to the first end 111 of the surrogate syringe barrel 110, but in other implementations, the first end 12 of the plunger rod 10 may be disposed at a distance within the body 112 of the surrogate syringe barrel 110.

By positioning the driving member 120 axially above the surrogate syringe barrel 110, the plunger rod 10, and the flange extender 20, the movement of the push pin 124 from the upper end 102 of the frame member 101 towards the lower end 103 of the frame member 101 simulates the force exerted on the first end 12 of the plunger rod 10 during expansion of any air or other gaseous bubbles within the actual syringe barrel of the drug delivery device during sterilization and/or transportation steps. The movable coupling 122 causes the push pin 124 to push the first end 12 of the plunger rod 10 in the downward direction while the load cell 121 measures this force, which progressively increases as the driving member 120 moves downward. As previously noted, the ring 16 of the plunger rod 10 engages the lower opening 24 of the flange extender 20 and temporarily retains the plunger rod 10 in this position until the urging force exerted by the movable coupling 122 is sufficient to overcome the retention force. The maximum value measured by the load cell 121 during this movement is herein referred to as the maximum urging force. Eventually, the ring 16 of the plunger rod 10 and/or the floor region 23 of the flange extender 20 will deform to the point that the ring 16 of the plunger rod 10 passes through the lower opening 24 of the flange extender 20 (FIG. 6). The load cell 121 measures the maximum urging force, which corresponds to a maximum retention force exhibited between the plunger rod 10 and the flange extender 20. This measurement may be an indication of the maximum internal pressure these components may withstand prior to exhibiting undesired relative translation.

The resulting data may be used to determine whether retention features of the combination of the tested plunger rod and flange extender generate sufficient interference to adequately withstand internal and/or external forces prior to drug administration. Depending on the specific desired drug, the quantity thereof in the syringe barrel, and/or any other additional variables, a maximum internal pressure may be calculated. This value may serve as a minimum threshold value (while accounting for any desired safety factors) that the desired plunger rod and flange extender must achieve to be used with the desired drug. Advantageously, any number of different plunger rods and/or flange extenders having varying characteristics (e.g., size, materials, features, etc.) may be tested during different stages of implementation (e.g., during a design or development phase, a design validation phase, and/or during manufacturing). Other examples are possible.

In some arrangements (see, e.g., FIGS. 2 & 3), the end of the push pin 124 may have a concave or similar interior profile that is arranged to receive a portion of the first end 12 of the plunger rod 10. In such examples, the push pin may have alignment features formed thereon to ensure the system 100 maintains the correct relative positioning between these components. Such an arrangement may allow the push pin 124 to apply a uniform downward force on the plunger rod 10, thereby further mimicking the force(s) occurring within the actual drug delivery device.

As compared with conventional testing systems which apply a pulling force on the plunger rod, the present testing system 100 pushes or urges the plunger rod 10 downwards, thus closely simulating both the magnitude and direction of forces experienced by the respective components during sterilization. In those conventional systems, the use of a pulling force applied to the plunger rod to measure plunger rod retention force could result in inaccurate measurements due to differences in component/system behavior when loaded in compression vs. tension (and/or any internal forces). Accordingly, the present system provides increased accuracy by closely simulating the pushing forces generated from the syringe barrel on the plunger rod and related components.

Further, the present testing system 100 provides an approach for retaining the syringe barrel above the lower end 103 of the frame member 101 to allow the plunger rod to translate downwards by providing ample clearance between upper and lower ends 102, 103 thereof. As previously noted, the testing system 100 may also advantageously be used to determine the retention force in other environments such as during transportation. Altitude changes during transportation may cause air bubbles inside the PFS to expand or compress to achieve an equilibrium volume, which may in turn exert different forces on the plunger stopper (and thus the plunger rod). This test measures the retention force to determine if it is sufficient to maintain the integrity of the drug container's sterile barrier, and also assists in ensuring components do not become disassembled.

The above description describes various devices, assemblies, components, subsystems and methods for use related to a drug delivery device. The devices, assemblies, components, subsystems, methods or drug delivery devices can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir. In some instances, the reservoir is a primary container that is either filled or pre-filled for treatment with the drug. The primary container can be a vial, a cartridge or a pre-filled syringe.

In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF), UDENYCA® (pegfilgrastim-cbqv), Ziextenzo® (LA-EP2006; pegfilgrastim-bmez), or FULPHILA (pegfilgrastim-bmez).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RANKL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“IL1-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 145c7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like; Hepatocyte growth factor (“HGF”) specific antibodies, peptibodies, related proteins, and the like, including those that target the HGF/SF: cMet axis (HGF/SF: c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa) Erythropoietin [30-asparagine, 32-threonine, 87-valine, 88-asparagine, 90-threonine], Darbepoetin alfa, novel erythropoiesis stimulating protein (NESP); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-α4ß7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Kanjinti™ (trastuzumab-anns) anti-HER2 monoclonal antibody, biosimilar to Herceptin®, or another product containing trastuzumab for the treatment of breast or gastric cancers; Humatrope® (somatropin, Human Growth Hormone); Humira® (adalimumab); Vectibix® (panitumumab), Xgeva® (denosumab), Prolia® (denosumab), Immunoglobulin G2 Human Monoclonal Antibody to RANK Ligand, Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Soliris™ (eculizumab); pexelizumab (anti-C5 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNFα monoclonal antibody); Reopro® (abciximab, anti-GP IIb/IIia receptor monoclonal antibody); Actemra® (anti-IL6 Receptor mAb); Avastin® (bevacizumab), HuMax-CD4 (zanolimumab); Mvasi™ (bevacizumab-awwb); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 145c7-CHO (anti-IL15 antibody, see U.S. Pat. No. 7,153,507); Tysabri® (natalizumab, anti-α4 integrin mAb); Valortim® (MDX-1303, anti-B. anthracis protective antigen mAb); ABthrax™; Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2Rα mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFα mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-α5β1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MYO-029); anti-GM-CSF Receptor mAb (CAM-3001); anti-HepC mAb (HuMax HepC); anti-IFNα mAb (MEDI-545, MDX-198); anti-IGF1R mAb; anti-IGF-1R mAb (HuMax-Inflam); anti-IL12 mAb (ABT-874); anti-IL12/IL23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCGβ mAb (MDX-1307); anti-mesothelin dsFv-PE38 conjugate (CAT-5001); anti-PD1mAb (MDX-1106 (ONO-4538)); anti-PDGFRα antibody (IMC-3G3); anti-TGFß mAb (GC-1008); anti-TRAIL Receptor-2 human mAb (HGS-ETR2); anti-TWEAK mAb; anti-VEGFR/Flt-1 mAb; and anti-ZP3 mAb (HuMax-ZP3).

In some embodiments, the drug delivery device may contain or be used with a sclerostin antibody, such as but not limited to romosozumab, blosozumab, BPS 804 (Novartis), Evenity™ (romosozumab-aqqg), another product containing romosozumab for treatment of postmenopausal osteoporosis and/or fracture healing and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TIMP-3. In some embodiments, the drug delivery device may contain or be used with Aimovig® (erenumab-aooe), anti-human CGRP-R (calcitonin gene-related peptide type 1 receptor) or another product containing erenumab for the treatment of migraine headaches. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. Additionally, bispecific T cell engager (BiTE®) molecules such as but not limited to BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure. In some embodiments, the drug delivery device may contain or be used with Avsola™ (infliximab-axxq), anti-TNF a monoclonal antibody, biosimilar to Remicade® (infliximab) (Janssen Biotech, Inc.) or another product containing infliximab for the treatment of autoimmune diseases. In some embodiments, the drug delivery device may contain or be used with Kyprolis® (carfilzomib), (2S)—N—((S)-1-((S)-4-methyl-1-((R)-2-methyloxiran-2-yl)-1-oxopentan-2-ylcarbamoyl)-2-phenylethyl)-2-((S)-2-(2-morpholinoacetamido)-4-phenylbutanamido)-4-methylpentanamide, or another product containing carfilzomib for the treatment of multiple myeloma. In some embodiments, the drug delivery device may contain or be used with Otezla® (apremilast), N-[2-[(1S)-1-(3-ethoxy-4-methoxyphenyl)-2-(methylsulfonyl)ethyl]-2,3-dihydro-1,3-dioxo-1H-isoindol-4-yl]acetamide, or another product containing apremilast for the treatment of various inflammatory diseases. In some embodiments, the drug delivery device may contain or be used with Parsabiv™ (etelcalcetide HCl, KAI-4169) or another product containing etelcalcetide HCl for the treatment of secondary hyperparathyroidism (sHPT) such as in patients with chronic kidney disease (KD) on hemodialysis. In some embodiments, the drug delivery device may contain or be used with ABP 798 (rituximab), a biosimilar candidate to Rituxan®/MabThera™, or another product containing an anti-CD20 monoclonal antibody. In some embodiments, the drug delivery device may contain or be used with a VEGF antagonist such as a non-antibody VEGF antagonist and/or a VEGF-Trap such as aflibercept (Ig domain 2 from VEGFR1 and Ig domain 3 from VEGFR2, fused to Fc domain of IgG1). In some embodiments, the drug delivery device may contain or be used with ABP 959 (eculizumab), a biosimilar candidate to Soliris®, or another product containing a monoclonal antibody that specifically binds to the complement protein C5. In some embodiments, the drug delivery device may contain or be used with Rozibafusp alfa (formerly AMG 570) is a novel bispecific antibody-peptide conjugate that simultaneously blocks ICOSL and BAFF activity. In some embodiments, the drug delivery device may contain or be used with Omecamtiv mecarbil, a small molecule selective cardiac myosin activator, or myotrope, which directly targets the contractile mechanisms of the heart, or another product containing a small molecule selective cardiac myosin activator. In some embodiments, the drug delivery device may contain or be used with Sotorasib (formerly known as AMG 510), a KRASG12C small molecule inhibitor, or another product containing a KRASG12C small molecule inhibitor. In some embodiments, the drug delivery device may contain or be used with Tezepelumab, a human monoclonal antibody that inhibits the action of thymic stromal lymphopoietin (TSLP), or another product containing a human monoclonal antibody that inhibits the action of TSLP. In some embodiments, the drug delivery device may contain or be used with AMG 714, a human monoclonal antibody that binds to Interleukin-15 (IL-15) or another product containing a human monoclonal antibody that binds to Interleukin-15 (IL-15). In some embodiments, the drug delivery device may contain or be used with AMG 890, a small interfering RNA (siRNA) that lowers lipoprotein(a), also known as Lp(a), or another product containing a small interfering RNA (siRNA) that lowers lipoprotein(a). In some embodiments, the drug delivery device may contain or be used with ABP 654 (human IgG1 kappa antibody), a biosimilar candidate to Stelara®, or another product that contains human IgG1 kappa antibody and/or binds to the p40 subunit of human cytokines interleukin (IL)-12 and IL-23. In some embodiments, the drug delivery device may contain or be used with Amjevita™ or Amgevita™ (formerly ABP 501) (mab anti-TNF human IgG1), a biosimilar candidate to Humira®, or another product that contains human mab anti-TNF human IgG1. In some embodiments, the drug delivery device may contain or be used with AMG 160, or another product that contains a half-life extended (HLE) anti-prostate-specific membrane antigen (PSMA)×anti-CD3 BiTE® (bispecific T cell engager) construct. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 133, or another product containing a gastric inhibitory polypeptide receptor (GIPR) antagonist and GLP-1R agonist. In some embodiments, the drug delivery device may contain or be used with AMG 171 or another product containing a Growth Differential Factor 15 (GDF15) analog. In some embodiments, the drug delivery device may contain or be used with AMG 176 or another product containing a small molecule inhibitor of myeloid cell leukemia 1 (MCL-1). In some embodiments, the drug delivery device may contain or be used with AMG 199 or another product containing a half-life extended (HLE) bispecific T cell engager construct (BiTE®). In some embodiments, the drug delivery device may contain or be used with AMG 256 or another product containing an anti-PD-1×IL21 mutein and/or an IL-21 receptor agonist designed to selectively turn on the Interleukin 21 (IL-21) pathway in programmed cell death-1 (PD-1) positive cells. In some embodiments, the drug delivery device may contain or be used with AMG 330 or another product containing an anti-CD33×anti-CD3 BiTE® (bispecific T cell engager) construct. In some embodiments, the drug delivery device may contain or be used with AMG 404 or another product containing a human anti-programmed cell death-1 (PD-1) monoclonal antibody being investigated as a treatment for patients with solid tumors. In some embodiments, the drug delivery device may contain or be used with AMG 427 or another product containing a half-life extended (HLE) anti-fms-like tyrosine kinase 3 (FLT3)×anti-CD3 BiTE® (bispecific T cell engager) construct. In some embodiments, the drug delivery device may contain or be used with AMG 430 or another product containing an anti-Jagged-1 monoclonal antibody. In some embodiments, the drug delivery device may contain or be used with AMG 506 or another product containing a multi-specific FAP×4-1BB-targeting DARPin® biologic under investigation as a treatment for solid tumors. In some embodiments, the drug delivery device may contain or be used with AMG 509 or another product containing a bivalent T-cell engager and is designed using XmAb® 2+1 technology. In some embodiments, the drug delivery device may contain or be used with AMG 562 or another product containing a half-life extended (HLE) CD19×CD3 BiTE® (bispecific T cell engager) construct. In some embodiments, the drug delivery device may contain or be used with Efavaleukin alfa (formerly AMG 592) or another product containing an IL-2 mutein Fc fusion protein. In some embodiments, the drug delivery device may contain or be used with AMG 596 or another product containing a CD3× epidermal growth factor receptor vIII (EGFRvIII) BiTE® (bispecific T cell engager) molecule. In some embodiments, the drug delivery device may contain or be used with AMG 673 or another product containing a half-life extended (HLE) anti-CD33×anti-CD3 BiTE® (bispecific T cell engager) construct. In some embodiments, the drug delivery device may contain or be used with AMG 701 or another product containing a half-life extended (HLE) anti-B-cell maturation antigen (BCMA)×anti-CD3 BiTE® (bispecific T cell engager) construct. In some embodiments, the drug delivery device may contain or be used with AMG 757 or another product containing a half-life extended (HLE) anti-delta-like ligand 3 (DLL3)×anti-CD3 BiTE® (bispecific T cell engager) construct. In some embodiments, the drug delivery device may contain or be used with AMG 910 or another product containing a half-life extended (HLE) epithelial cell tight junction protein claudin 18.2×CD3 BiTE® (bispecific T cell engager) construct.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

1. A testing system for a drug delivery device comprising:
a frame member having a first end and a second end;
a driving member positioned near the frame member, the driving member being movable in an axial direction between the first end of the frame member and the second end of the frame member;
a surrogate syringe barrel operably coupled with the of the frame member;
a flange extender adapted to couple with a portion of the surrogate syringe barrel; and
a plunger rod adapted to couple with the flange extender and be at least partially disposed within the surrogate syringe barrel while being movable therein, the plunger rod including a first end, a second end, and a longitudinal length therebetween, the first end being positioned near a portion of the driving member;
wherein the driving member is adapted to measure a maximum urging force exerted on the plunger rod when moving in the axial direction towards the second end of the frame member.

2. The testing system of claim 1, wherein the plunger rod includes a plunger rod interface positioned along the longitudinal length thereof, the plunger rod interface adapted to engage a floor region of the flange extender to retain the plunger rod in an initial position.

3. The testing system of claim 2, wherein the plunger rod interface includes a ring having an outer dimension.

4. The testing system of claim 2, wherein the floor region of the flange extender includes an opening having an inner dimension.

5. The testing system of claim 4, wherein the outer dimension of the ring of the plunger rod is greater than the inner dimension of the opening of the flange extender such that axial movement of the plunger rod towards the second end of the frame member is prevented.

6. The testing system of claim 2, wherein at least one of the plunger rod interface or the floor region of the flange is at least partially constructed from a deformable material.

7. The testing system of claim 1, wherein the surrogate syringe barrel includes a syringe flange, wherein the flange extender includes a flange adapted to rest upon the syringe flange of the surrogate syringe barrel.

8. The testing system of claim 1, wherein the frame member includes an elevated platform positioned at the first end thereof, the elevated platform adapted to retain and suspend the surrogate syringe barrel from the first end thereof.

9. The testing system of claim 8, wherein when moving the driving member towards the second end of the frame member, the plunger rod and the flange extender are subject to a gravitational force in the axial direction.

10. The testing system of claim 1, wherein the driving member includes a push pin adapted to be at least partially insertable and movable within the surrogate syringe barrel.

11. The testing system of claim 10, wherein the push pin includes a recessed region adapted to align with the first end of the plunger rod.

12. The testing system of claim 1, wherein the driving member is positioned above the first end of the frame member.

13. A method of measuring a maximum urging force experienced by a portion of a drug delivery device, the method comprising:
positioning a driving member near a first end of a frame member, the driving member being movable in an axial direction between the first end of the frame member to a second end of the frame member;
operably coupling a surrogate syringe barrel with the first end of the frame member;
coupling a first flange extender with a portion of the surrogate syringe barrel;
coupling a first plunger rod with the flange extender such that the first plunger rod is at least partially movably disposed within the surrogate syringe barrel, the first plunger rod including a first end, a second end, and a longitudinal length therebetween;
moving the driving member in the axial direction towards the second end of the frame member, thereby urging the first plunger rod towards the second end of the frame member; and
measuring a maximum urging force exerted on the first plunger rod while moving towards the second end of the frame member.

14. The method of claim 13, wherein the plunger rod includes a plunger rod interface positioned along the longitudinal length thereof, the plunger rod interface adapted to engage a floor region of the flange extender to retain the plunger rod in an initial position.

15. The method of claim 13, further comprising the step of resting a flange of the flange extender on a syringe flange of the surrogate syringe barrel.

16. The method of claim 13, further comprising the step of retaining and suspending the surrogate syringe barrel from an elevated platform of the frame member positioned at the first end thereof.

17. The method of claim 16, wherein the step of moving the driving member towards the second end of the frame member further comprises subjecting the plunger rod and the flange extender to a gravitational force in the axial direction.

18. The method of claim 13, wherein the step of moving the driving member in the axial direction comprises moving a push pin within the surrogate syringe barrel.

19. The method of claim 18, further comprising the step of aligning a recessed region of the push pin with the first end of the plunger rod.
